(19) United States
(12) Patent Application Publication
(10) Pub. No.: US 2006/0188937 A1
(43) Pub. Date: Aug. 24, 2006

(54) G-protein estrogen membrane receptor

(75) Inventors: Peter Thomas, Portland, TX (US); Yefei Pang, Corpus Christi, TX (US); Edward Filardo, Providence, RI (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 11/252,084

(22) Filed: Oct. 17, 2005

Related U.S. Application Data
(60) Provisional application No. 60/620,188, filed on Oct. 19, 2004.

(51) Int. Cl. G01N 33/53; C07H 21/04; C12P 21/06; C07K 14/72

(52) U.S. Cl. 435/7.1; 435/69.1; 435/320.1; 435/325; 530/350; 536/23.5

(57) ABSTRACT

The present invention includes compositions and methods for the identification, isolation and characterization of a G-protein-coupled receptor that binds to an estrogen or estrogen-like molecule, to modulators of the receptor's activity and diagnostic methods for use in detection and treatment of a G-protein-coupled receptor related cancer.

This patent application claims priority to U.S. Provisional Patent Application Ser. No. 60/620,188, filed Oct. 19, 2004, the entire contents of which are incorporated herein by reference.

This invention was made with U.S. Government support under Contract No. EPA STAR Grant R-82902401. The government has certain rights in this invention. Without limiting the scope of the invention, its background is described in connection with estrogen receptors.

### TECHNICAL FIELD OF THE INVENTION

The present invention relates in general to the field of steroid receptors, and more particularly, it provides compositions and methods for using a novel G-protein estrogen membrane receptor.

### BACKGROUND OF THE INVENTION

Although nonclassical estrogen actions initiated at the cell surface have been described in many tissues, the identities of the membrane estrogen receptors (mERs) mediating these actions remain unclear. The classic genomic mechanism of steroid action is mediated by intracellular receptors belonging to the nuclear steroid receptor superfamily. There is now convincing evidence that steroids also exert rapid, nongenomic steroid actions initiated at the cell surface by binding to membrane receptors (1-3).

### SUMMARY OF THE INVENTION

The present inventors have recognized that despite intensive research, the identities of steroid membrane receptors remain unclear and surrounded by controversy (4-6). Investigations of nonclassical estrogen signaling suggest nuclear estrogen receptors, ERα and ERβ or ER-like proteins are likely candidates for the membrane estrogen receptors (mERs) mediating these estrogen actions in a variety of target cells, including endothelial, neuronal and pituitary cells (7-11). However, evidence has also been obtained for the involvement of novel mERs unrelated to nuclear ERs in nonclassical estrogen actions in several other cell types, many of which are associated with G-proteins (12-16).

The present inventors have discovered a novel G-protein-coupled receptor that binds to estrogen and is associated with the cell membrane. More particularly, it has been found that GPR30, an orphan receptor unrelated to nuclear estrogen receptors (nERs), has all the binding and signaling characteristics of a mER. A high affinity (Kd: 2.7 nM), limited capacity, displaceable, single binding site specific for estrogens was detected in plasma membranes of SKBR3 breast cancer cells that express GPR30, but lack nERs. Progesterone-induced increases and siRNA-induced decreases in GPR30 expression in SKBR3 cells were accompanied by parallel changes in specific estradiol-17β (E2) binding. Plasma membranes of HEK293 cells transfected with GPR30, but not those of untransfected cells, and human placental tissues that express GPR30, also displayed high affinity, specific estrogen binding typical of mERs. E2 treatment of transfected cell membranes caused activation of a stimulatory G-protein (Gₛ) that is directly coupled to the receptor, indicating GPR30 is a G-protein coupled receptor (GPCR), which also increased adenylyl cyclase activity. The finding that the anti-estrogens tamoxifen and ICI 182,780, and an environmental estrogen, o,p′-DDE, have high binding affinities to the receptor and mimic the actions of E2 has important implications for both the development and treatment of estrogen-dependent breast cancer.

Recently, the present inventors discovered a novel family of GPCR-like membrane progestin receptors (mPRs) that are unrelated to GPR30. The identification of a second distinct class of GPCR-like steroid membrane receptors suggests a widespread role for GPCRs in nonclassical steroid hormone actions. These hitherto unknown family of mPRs, unrelated to nuclear steroid receptors, but instead with characteristics of GPCRs (17, 18), prompted the inventors to search for other GPCRs with characteristics of steroid membrane receptors.

The orphan GPCR-like protein, GPR30, is widely distributed in neural, breast cancer, placental, heart, ovarian, prostate, hepatic, vascular epithelial and lymphoid tissues, and shows structural sequence homology to receptors for angiotensin, interleukin, and a variety of chemokines, suggesting it may be a peptide receptor (19-22). However, a broad range of chemotactic peptides and angiotensins showed no binding affinity for GPR30 (20, 23). Instead evidence was obtained for an involvement of GPR30 in estrogen-induced transactivation of epidermal growth factor receptor and adenylyl cyclase activity in SKBR3 breast cancer cells that lack nuclear estrogen receptors (24-26), suggesting GPR30 may be a novel mER. The present results demonstrate that expression of GPR30 in cells lacking ERα and ERβ is associated with the presence of high affinity, limited capacity and specific E2 binding to their plasma membranes characteristic of mERs. Evidence is presented that GPR30 is directly coupled to a stimulatory G-protein to upregulate adenylyl cyclase activity and is a GPCR.

More particularly, the present invention includes an isolated and purified G-protein-coupled receptor that binds specifically to an estrogen. The G-protein receptor may be a GPR30, e.g., a human GPR30. The G-protein receptor polypeptide may be a fusion protein, e.g., an isolated G-protein receptor polypeptide is a fusion protein comprising a myc-tag, a His-tag, FLAG-tag, Glutathione-S-Transferase, Maltose-Binding Protein or combinations thereof. It has been found that the G-protein receptor of the present invention triggers a non-classical estrogen signaling, e.g., the G-protein receptor disclosed herein binds to estradiol-17β (E2). The G-protein receptor may be isolated from neural, breast cancer, placental, heart, ovarian, prostate, hepatic, vascular epithelial and lymphoid tissues.

More particularly, the present invention includes an isolated and purified G-protein coupled estrogen receptor polypeptide encoded by the nucleic acid fragment of SEQ ID NO.:1, GenBank; accession No. BC011634, the polypeptide having the sequence of SEQ ID NO.: 2.

The present invention also includes a method for identifying a test compound that modulates the binding of an estrogen to a G-protein-coupled receptor by measuring the binding of an estrogen to a G-protein receptor to the estrogen in the presence and absence of a test compound, wherein the test compound modulates the binding of the estrogen to the G-protein receptor and is indicative that the test compound is a modulator of the binding of the estrogen to the G-protein receptor. Examples of such steroids include: 17β-estradiol (E2), 17α-estradiol (E2α), estrone (E2), estriol (E3), cortisol (cor), testosterone (T) and progesterone (P4) and the synthetic estrogen diethylstilbestrol (DES). Other compounds for use in the method disclosed herein include: tamoxifen (Tmx), zearalenone, dichlorodiphenyltrichloroethane, o,p′-DDE (DDE), the synthetic antiestrogen ICI 182, 780, a taxol-derivative or salts thereof.

Yet another embodiment of the present invention includes a vector that includes a nucleic acid sequence of SEQ ID NO.:1 and conserved variants thereof, GenBank; accession No. BC011634, a host cell with a vector that includes the nucleic acid sequence of SEQ ID NO.:1, GenBank; accession No. BC01 1634.

The present invention may be used in a method for treating cancer by identifying a patient in need of cancer therapy and providing to the patient an effective dose of a GPCR modulator. In certain embodiments, the method may be used to screen patients in a clinical trial concurrent with or prior to entering them in the trial. This method may be used to pre-screen those patients that will have an untoward reaction to the trial, thereby improving the potential outcome of the trail and reducing the possibility for harm to the patient. The PCR modulator may be a GPCR agonist, a GPCR antagonist and/or an agonist or antagonist of a GTPase activity of the GPCR.

Another embodiment of the present invention is a method of diagnostic a condition related to nonclassical estrogen binding that includes the step of binding a GPCR binding agent comprising a detectable label to a cell, e.g., a GPCR binding agent that is specific for membrane estrogen binding activity. A dosage form may be a therapeutically effective amount of an estrogen or estrogen derivative that is specific for a GPCR, e.g., an estrogen modulator, an estrogen or estrogen derivative and/or a GPCR agonist.

Alternatively, the present invention also includes a method of identifying a GCPR modulator by screening a compound library for one or more agents that bind to a membrane-associated G-protein estrogen receptor. The method may also include the step of determining if the one or more agents that bind to the membrane-associated G-protein receptor is selective for the membrane-associated G-protein estrogen receptor. For use with the invention, an isolated and purified membrane-associated G-protein estrogen receptor will finds particular uses. The isolated and purified membrane-associated G-protein estrogen receptor may be used in a diagnostic method for characterizing the expression of an isolated and purified membrane-associated G-protein estrogen receptor of a patient, followed by treating the patient with an agent that modifies the activity of the membrane-associated G-protein estrogen receptor. Finally, the present invention may be used to identify the expression of the isolated and purified membrane-associated G-protein estrogen receptor and variants thereof using an antibody that detects the isolated and purified membrane-associated G-protein estrogen receptor and/or fusion proteins thereof.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F show the estrogen binding characteristics of plasma membranes from SKBR3 cells (ERα−, ERβ−, GPR30+).

FIGS. 2A to 2E show that estrogen binds to plasma membranes of HEK293 cells (ERα−, ERβ−) stably transfected with GPR30.

FIGS. 3A to 3F show the coupling of GPR30 to G-proteins and activation of adenylyl cyclase in SKBR3 and transfected HEK293 cells.

FIGS. 4A to 4F show modulation of binding of E2 and GPR30 expression by cholera toxin and hormone treatments and GPR30 expression and detection of GPR30 and E2 binding in human placental tissues.

FIGS. 5A and 5B show no detection of nuclear ER mRNA and protein in the SKBR3 cells and immunocytochemical staining of cells with GPR30 antibody.

FIGS. 6A and 6B show no detection of nuclear ER mRNA and protein in the HEK293 cells and immunocytochemical staining of cells with GPR30 antibody.

FIGS. 7A, 7B and 7C shows no detection of GPR30 in some other estrogen responsive cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used throughout the present specification the following abbreviations are used: TF, transcription factor; ORF, open reading frame; kb, kilobase (pairs); UTR, untranslated region; kD, kilodalton; PCR, polymerase chain reaction; RT, reverse transcriptase.

The terms “a sequence essentially as set forth in SEQ ID NO. (#)”, “a sequence similar to”, “nucleotide sequence” and similar terms, with respect to nucleotides, refers to sequences that substantially correspond to any portion of the sequence identified herein as SEQ ID NO.: 1. These terms refer to synthetic as well as naturally-derived molecules and includes sequences that possess biologically, immunologically, experimentally, or otherwise functionally equivalent activity, for instance with respect to hybridization by nucleic acid segments, or the ability to encode all or portions of a G-protein coupled membrane estrogen receptor and proteins with equivalent activities. Naturally, these terms are meant to include information in such a sequence as specified by its linear order.

The term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive PCR assay) and is meant, as will be known to those of skill in the art, to include specific interaction even at low stringency.

The inhibition of hybridization of the completely complementary sequence to the target sequence may also be examined using a hybridization assay involving a solid support (e.g., Southern or Northern blot, solution hybridization and the like) under conditions of low stringency. Low stringency conditions may be used to identify the binding of two sequences to one another while still being specific (i.e., selective). The absence of non-specific binding may be tested by the use of a second target that lacks even a partial degree of complementarity (e.g., less than about 30% identity). In the absence of non-specific binding, the probe will not hybridize to the second non-complementary target and the original interaction will be found to be selective. Low stringency conditions are generally conditions equivalent to binding or hybridization at 42 degrees Centigrade in a solution consisting of 5× SSPE (43.8 g/l NaCl, 6.9 g/l NaH2PO4-H2O and 1.85 g/l EDTA, pH 7.4), 0.1% SDS, 5× Denhardt's reagent (50× Denhardt's contains per 500 ml: 5 g Ficoll (Type 400, Pharmacia), 5 g BSA (Fraction V; Sigma) and 100 micrograms/ml denatured salmon sperm DNA); followed by washing in a solution comprising 5× SSPE, 0.1% SDS at 42 degrees Centigrade when a probe of about 500 nucleotides in length is employed. The art knows that numerous equivalent conditions may be employed to achieve low stringency conditions. Factors that affect the level of stringency include: the length and nature (DNA, RNA, base composition) of the probe and nature of the target (DNA, RNA, base composition, present in solution or immobilized, etc.) and the concentration of the salts and other components (e.g., formamide, dextran sulfate, polyethylene glycol). Likewise, the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, inclusion of formamide, etc.).

An oligonucleotide sequence that is “substantially homologous” to the G-protein coupled membrane estrogen receptor of SEQ ID NO:1″ is defined herein as an oligonucleotide sequence that exhibits greater than or equal to 75%, 80%, 85%, 90%, 95% identity to the sequence of SEQ ID NO:1 when sequences having a length of 100 bp or larger are compared.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” is used in reference to nucleic acid molecules that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome

The term “host cell” refers to cells that have been engineered to contain nucleic acid segments or altered segments, whether archeal, prokaryotic, or eukaryotic. Thus, engineered, or recombinant cells, are distinguishable from naturally occurring cells that do not contain recombinantly introduced genes through the hand of man.

The term “agonist” refers to a molecule that enhances either the strength or the time of an effect of G-protein coupled membrane estrogen receptor and encompasses small molecules, proteins, nucleic acids, carbohydrates, lipids, or other compounds. The term “antagonist” refers to a molecule that decreases either the strength or the time of an effect of G-protein coupled membrane estrogen receptor and encompasses small molecules, proteins, nucleic, acids, carbohydrates, lipids, or other compounds.

The term “altered”, or “alterations” or “modified” with reference to nucleic acid or polypeptide sequences is meant to include changes such as insertions, deletions, substitutions, fusions with related or unrelated sequences, such as might occur by the hand of man, or those that may occur naturally such as polymorphisms, alleles and other structural types. Alterations encompass genomic DNA and RNA sequences that may differ with respect to their hybridization properties using a given hybridization probe. Alterations of polynucleotide sequences for the G-protein coupled membrane estrogen receptor, or fragments thereof, include those that increase, decrease, or have no effect on functionality. Alterations of polypeptides refer to those that have been changed by recombinant DNA engineering, chemical, or biochemical modifications, such as amino acid derivatives or conjugates, or post-translational modifications.

As used herein, the term “vector” is used in reference to nucleic acid molecules that transfer DNA segment(s) from one cell to another, in the present case a G-protein coupled membrane estrogen receptor. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “amplify”, when used in reference to nucleic acids refers to the production of a large number of copies of a nucleic acid sequence by any method known in the art. Amplification is a special case of nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted oat from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

A dosage unit for use an agonist or antagonist of the G-protein coupled membrane estrogen receptor of the present invention, may be a single compound or mixtures thereof with other compounds. The compounds may be mixed together, form ionic or even covalent bonds. The agonist or antagonist of the G-protein coupled membrane estrogen receptor of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide agonists or antagonists of the G-protein coupled membrane estrogen receptor of the present invention to a patient in need of therapy. For example, selective antagonists or agonists may be selected from known estrogen and estrogen derivatives based on their relative interaction with the intracellular estrogen receptor verses the G-protein coupled membrane estrogen receptor and/or combinations with G-protein agonists or antagonists and combinations thereof. The compounds may be administered as any one of known salt forms.

Agonist or antagonist of the G-protein coupled membrane estrogen receptor are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the membrane estrogen receptor agonist or antagonist may be formulated to provide, e.g., maximum and/or consistent dosing for the particular form for oral, rectal, topical, intravenous injection or parenteral administration. While the agonist or antagonist of the G-protein coupled membrane estrogen receptor may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions incorporated herein by reference.

For example, the agonist or antagonist of the G-protein coupled membrane estrogen receptor may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The agonist or antagonist of the G-protein coupled membrane estrogen receptor may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like. Alternatively, the compounds may be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the agonist or antagonist of the G-protein coupled membrane estrogen receptor may be coupled one or more biodegradable polymers to achieve controlled release of the compound, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the one or more agonists/antagonists and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagous, tachea, lungs and alveoli, the agonist or antagonist of the G-protein coupled membrane estrogen receptor may also be delivered as an intranasal form via use of a suitable intranasal vehicle. For dermal and transdermal delivery, the steroid may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of agonist or antagonist of the G-protein coupled membrane estrogen receptor may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of agonist or antagonist of the G-protein coupled membrane estrogen receptor. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Materials and Methods. Chemicals. The steroids 17β-estradiol (E2), 17α-estradiol (E2α), estrone (E2), estriol (E3), cortisol (cor), testosterone (T) and progesterone (P4) and the synthetic estrogen diethylstilbestrol (DES) were purchased from Steraloids (Newport, R.I.). The antiestrogen tamoxifen (Tmx) and the fungal metabolite, zearalenone, were purchased from Sigma-Aldrich Corp. (St. Louis, Mo.). The derivative of the pesticide dichlorodiphenyltrichloroethane, o,p′-DDE (DDE), was purchased from Chem Service (West Chester, Pa.). The synthetic antiestrogen ICI 182, 780 (ICI) was purchased from Tocris (Ellisvilee, Mo.). 17β-[2,4,6,7-³H]-estradiol ([³H]E2); ˜89 Ci/mmol, was purchased from Amersham Pharmacia Biotech (Piscataway, N.J.). All other chemicals, buffers and media were purchased form Sigma-Aldrich unless noted otherwise.

Cell culture and transfections. Human SKBR3 and HEK293 cells (American Type Culture Collection, Manassas, Va.) were cultured in DMEM/Ham's F-12 medium without phenol red supplemented with 10% fetal bovine serum (FBS) and 100 μg/ml of gentamicin, with changes of medium every 1-2 days. SKBR3 cells were transiently transfected with GPR30 siRNA (100 nm), or nonspecific, pre-synthesized siRNA (control) using Lipofectamine 2000 (Life Technologies Inc., Gaithersburg, Md.) at 25° C., following the manufacturer's procedures (Dharmacon, Layfayette, Colo.) to interfere with GPR30 expression, and experiments were conducted 18 hr later. HEK293 cells were transfected with a GPR30 construct, consisting of the full-length cDNA ligated into the pBK-CMV expression vector (25), using Lipofectamine 2000 and grown to confluence. Geneticin (500 μg/ml) was added and the geneticin-resistant cells containing the GPR30 construct were propagated to generate stable cell lines (selectively maintained with 500 μg/ml geneticin). Cells reached 80% confluence after 3 days in culture (˜2×10⁸ cells, 0.6 mg cell membrane protein/150 mm dish) and were replaced with fresh media containing 0 or 5% FBS one day before experiments.

Treatments. GPR30 expression and receptor binding were upregulated in SKBR3 cells by incubating them for 16 hr in FBS-free media with 200 nm progesterone (P4) or E2, or media alone, followed by repeated washes with buffer before measurement of E2 binding. The effects of uncoupling G-proteins on E2 binding affinity was investigated with membranes of transfected HEK293 cells pretreated with 0 or 25 μM GTPγS at 25° C. for 30 min. Cells were also incubated with 10 μg/ml activated cholera toxin (activated with 4 mM DTT), inactive cholera toxin (inactivated by boiling) or media alone for 30 min at 37° C. immediately before preparation of the cell membrane for assay of E2 binding. Cells were collected with a cell scraper and washed twice with fresh media prior to preparation of plasma membranes. All studies were repeated at least three times with different batches of cultured cells.

Membrane preparation and solubilization. Plasma membrane fractions of human tissues and cells were obtained following homogenization and centrifugation procedures described previously (27, 28). Placental tissue plasma membranes were further purified by centrifuging the membrane pellet with a sucrose pad (1.2M sucrose) at 6,500×g for 45 min (17, 29). Membranes were solubilized with 12 mM Triton X-100 in four volumes HEPES buffer (25 nm HEPES, 10 mM NaCl, 1 mM dithioerythritol, DTT) for 30 min. followed by removal of the detergent with polystyrene adsorbants (2:1 vol:wt; Bio-Rad SM-2) and subsequent removal of the adsorbants by filtration (G-8 filter, Fisher) before the addition of loading buffer for Western blot analyses (17).

Estrogen receptor binding assays. General procedures used in our laboratory for assaying saturation, association and dissociation kinetics, and steroid specificity of ligand binding to steroid membrane receptors (27-29) were used to measure [³H]E2 binding to plasma membrane preparations. For saturation analysis, one set of tubes contained a range (0.5-8.0 nM) of [2,4,6,7-³H]E2 ([³H]E2, ˜89 Ci/mmol) alone (total binding) and another set also contained 100-fold excess (50-800 nM) E2 competitor (nonspecific binding). For competitive binding assays tubes contained 4 nM [³H]E2 and the steroid competitors (concentration range: 1 nM-100 μM; dissolved in 5 μl ethanol, 1% of the total volume which does not affect [³H]E2 binding in the assay). After a 30-min incubation at 4° C. with the membrane fractions, the reaction was stopped by filtration (Whatman GF/B filters), the filters were washed and bound radioactivity measured by scintillation counting. The displacement of [³H]E2 binding by the steroid competitors was expressed as a percentage of the maximum specific binding of E2. Each assay point was run in triplicate and the assays were repeated utilizing different batches of cultured cells for each test chemical.

Western blot analysis. Solubilized membrane proteins were resolved by electrophoresis and western blot analysis performed as described previously (17), using an anti-GPR30 polyclonal antibody generated against a C-terminal 19 amino acid peptide fragment (24) (dilution: 1:1000) in an overnight incubation. The membrane was blocked with 5% nonfat milk in a TBST (50 mM Tris/100 mM NaCl/0.1% Tween 20, pH 7.4) buffer for 1 hr prior to incubation with the GPR30 antibody. The membrane was subsequently washed several times and then incubated for 1 hr at room temperature with horseradish peroxidase conjugated to goat anti-rabbit antibody (Cell Signaling), and visualized by treatment with enhanced chemiluminescence substrate (SuperSignal, Pierce, Rockford, Ill.).

cAMP measurement. Plasma membranes (1.5 mg/ml) were incubated in buffer (20 mM KCl, 12 mM MgCl₂, 3 mM EDTA, 2 mM ATP, 0.2 mM DTT, 10 mM creatine phosphate, 1 unit creatine kinase, 1 unit of pyruvate kinase and 20 mM HEPES, pH 7.5) with or without 100 nM of the test compounds for 20 minutes at 25° C. A standard concentration of 100 nm was chosen for comparison of the effects of compounds with low binding affinities for the receptor, although E2 has previously been shown to be effective in SKBR3 cells at a much lower concentration, 1 nm (25). Activated cholera toxin (10 μg/ml) was co-incubated with 100 nm E2 in some studies. The reaction was terminated by boiling the samples for 10 min. cAMP concentrations were measured in cytosolic fractions using an EIA kit following the manufacturer's instructions (Cayman Chemical, Ann Arbor, Mich.).

[³⁵S]GTPγ-S binding to cell membranes. Binding of [³⁵S]GTPγ-S to plasma membranes (˜10 μg protein) was assayed following the procedure of Liu and Dillon (30) with few modifications. Plasma membranes were incubated with 10 μM GDP and 0.5 nm [³⁵S]GTPγ-S (˜12,000 cpm, 1.0 Ci/mol) in 250%1 Tris buffer (100 nm NaCl, 5 mM MgCl₂, 1 mM CaCl₂, 0.6 mM EDTA, 0.1% BSA, 50 mM Tris-HCl, pH 7.4) at 25° C. for 30 min in the presence of 100 nm of the test compounds. Nonspecific binding was determined by addition of 100 μM GTPγ-S. At the end of the incubation period 100 μl aliquots were filtered through Whatman GF/B glass fiber filters, followed by several washes and subsequent scintillation counting.

Immunoprecipitation of [³⁵S]GTPγ-S-labeled G-protein α subunits. Immunoprecipitation of the G-protein alpha subunits coupled to [³⁵S]GTPγ-S was performed as described in (30). Breifly, plasma membranes (˜20 μg protein) of transfected HEK293 cells were incubated with 1 μM E2 for 30 min at 25° C. in 250 μl Tris buffer containing 4 nm [35 S]GTPγ-S, 10 μM GDP and protease inhibitor cocktail (Sigma-Aldrich, St. Louis, Mo.). The incubation was stopped by addition of 750 μl ice-cold buffer containing 100 μM GDP and 100 μM unlabelled GTPγ-S. Samples were centrifuged at 20,000×g for 15 min and the pellet resuspended in immunoprecititation buffer containing 1% Triton X-100, 0.1% SDS, 150 mM NaCl, 5 mM EDTA, 25 mM Tris-HCl, pH 7.4 and protease inhibitors. Specific antisera to the α subunits of G-proteins (Gᵢ, Gs, Sigma-Aldrich, dilution 1:500) were incubated with the samples for 6 hr at 4° C. Protein A-Sepharose was added and after an overnight incubation the immunoprecipitates were collected by centrifugation (12,000×g for 2 min) and washed in buffer (50 mM HEPES, 100 μM NaF, 50 mM sodium phosphate, 100 mM NaCl, 1% Triton X-100 and 1% SDS). The pellets were boiled in 0.5% SDS and the radioactivity in the immunoprecipitated [³⁵S]GTPγ-S-labeled G-protein α subunits counted.

RT-PCR of GPR30. Total RNA was extracted with Tri-reagent (Sigma-Aldrich) Reverse transcription was performed by adding 1-3 μg of RNA to the 10 μl reaction mix containing 1× first strand buffer (10 mM dithiothreitol (DTT), 0.5 mM of each dNTPs, 50 ng/μl oligo-dT primer and 100 U of Superscript II reverse transcriptase (Invitrogen, Carlsbad, Calif.), and the mixture was incubated for 2 hours at 42° C. The PCR reaction was conducted in 30 μl of PCR SuperMix (Invitrogen Corporation, Carlsabad, N. Mex.) that included 0.5 μl of the RT reaction and 0.2 μM of each of the primers. Gene specific primers for GPR30 (1.sense: 5′-GGC TTT GTG GGC AAC ATC-3′; antisense: 5′-CGG AAA GAC TGC TTG CAG G-3′; 2.sense: 5′-TGG TGG TGA ACA TCA GCT TC-3′, antisense: 5′-TGA GCT TGT CCC TGA AGG TC-3′; 3.sense: 5′-GCA GCG TCT TCT TCC TCA CC-3′; antisense: 5′-ACA GCC TGA GCT TGT CCC TG-3′); were designed according to the GPR30 sequence from GenBank; accession No. BC011634, relevant sequence incorporated herein by reference (Strausberger, et al., Generation and initial analysis of more than 15,000 full-length human and mouse cDNA sequences, Proc. Natl. Acad. Sci. U.S.A. 99 (26), 16899-16903 (2002). After an initial denaturation for 5 min at 94° C., the PCR reaction was performed on the Eppendorf Mastercycler for 35 cycles with the cycling profile of 30 s at 94° C., 30 s at 55° C., and 2 min at 72° C. followed by a 10-min extension at 72° C. The PCR reaction (5 μl) was electrophoresed on an agarose gel (1%) containing ethidium bromide to visualize the products. For semiquantitative RT-PCR 25 cycles of PCR reactions were performed (linear portion of cycle/product curve).

| SEQ ID NO:1 | |
|---|---|
| 1 | ggcacggagg ctttctaaag atggattcac catttaaaac agagctctgg gagcctttcg |
| 61 | gcaaatcttg aaagctgcac ggcgcagaga catggatgtg acttcccaag cccggggcgt |
| 121 | gggcctggag atgtacctag gcaccgcgca gcctgcggcc cccaacacca cctcccccga |
| 181 | gctcaacctg tcccacccgc tcctgggcac cgccctggcc aatgggacag gtgagctctc |
| 241 | ggagcaccag cagtacgtga tcggcctgtt cctctcgtgc ctctacacca tcttcctctt |
| 301 | ccccatcggc tttgtgggca acatcctgat cctggtggtg aacatcagct tccgcgagaa |
| 361 | gatgaccatc cccgacctgt acttcatcaa cctggcggtg gcggacctca tcctggtggc |
| 421 | cgactccctc attgaggtgt tcaacctgca cgagcggtac tacgacatcg ccgtcctgtg |
| 481 | caccttcatg tcgctcttcc tgcaggtcaa catgtacagc agcgtcttct tcctcacctg |
| 541 | gatgagcttc gaccgctaca tcgccctggc cagggccatg cgctgcagcc tgttccgcac |
| 601 | caagcaccac gcccggctga gctgtggcct catctggatg gcatccgtgt cagccacgct |
| 661 | ggtgcccttc accgccgtgc acctgcagca caccgacgag gcctgcttct gtttcgcgga |
| 721 | tgtccgggag gtgcagtggc tcgaggtcac gctgggcttc atcgtgccct tcgccatcat |
| 781 | cggcctgtgc tactccctca ttgtccgggt gctggtcagg gcgcaccggc accgtgggct |
| 841 | gcggccccgg cggcagaagg cgctccgcat gatcctcgcg gtggtgctgg tcttcttcgt |
| 901 | ctgctggctg ccggagaacg tcttcatcag cgtgcacctc ctgcagcgga cgcagcctgg |
| 961 | ggccgctccc tgcaagcagt ctttccgcca tgcccacccc ctcacgggcc acattgtcaa |
| 1021 | cctcgccgcc ttctccaaca gctgcctaaa ccccctcatc tacagctttc tcggggagac |
| 1081 | cttcagggac aagctgaggc tgtacattga gcagaaaaca aatttgccgg ccctgaaccg |
| 1141 | cttctgtcac gctgccctga aggccgtcat tccagacagc actgagcagt cggatgtgag |
| 1201 | gttcagcagt gccgtgtaga cagccttggc cacataggac cagccagggt gtgactcggg |
| 1261 | agctgcacac acctgggtgg acacaaggca cggccacgtc atgtctctaa actgcggtca |
| 1321 | gatgtggctt ctggctcctc ggggcctcgc gagggtcacg cttgcctggt caccctgggg |
| 1381 | ctgcttagga aacctcacga ctggtcacct tgcactcctc acacagaatt gctacaatcc |
| 1441 | caaagcgctc gccccgcagg gtccaaaggc cagcggtgac cagcctgtca cccagctcct |
| 1501 | ccccgccaac cctgcctgcc gctgcacctg cctgccgctg caggaaacat ttctgacacc |
| 1561 | gtcgaccagg aaagccacac ggagaggcca ctgtgggtga agcgcctcag ttacacagga |
| 1621 | accctaaagc aaatctgcca ccgtggggga actgacgctg gagatgcaag gtgctggtgg |
| 1681 | gtctgagctg gacgtcgcgg tgtgtcctct gtgcccacgg tctgagctag ctagcgcacc |
| 1741 | gccgagttaa agaggagaag gaaaacatgc tgctctggtg cacgcctgag cgtcctccat |
| 1801 | cttccaggat ggcagcaatg gcgctgtgcg gcctcaccag gcccacgagg agcagcagcg |
| 1861 | ctcggcccgg agcagcagga aggcccctct gtggagcgcc cgccgtctgc tccggggtgg |
| 1921 | ttcagtcact gcttgttgac atcaacatgg caattgcact catgtggact gggaccgtgc |
| 1981 | gagctgccgt gtgggttagt cgggtgccag gacaatgaaa tactccagca cgtgtggctg |
| 2041 | acgaatttgt ttctacagaa ataacagctg gggacaactg cggtgatgat gtaaaaacct |
| 2101 | tcccataaaa tgtaagaaaa gctgatgagg ctggtgacgt tcagcctttg tcaataaacc |
| 2161 | tgtcatgtgc ggataaaaaa aaaaaaaaaa aaaaaaaaaa aaaaaaaaaa aaaaaaaa |

SEQ ID NO.:2
MDVTSQARGVGLEMYLGTAQPAAPNTTSPELNLSHPLLGTALANGTGELSEHQQYVIGLFLSCLYTIF
LFPIGFVGNILILVVNISFREKMTIPDLYFINLAVADLILVADSLIEVFNLHERYYDIAVLCTFMSLF
LQVNMYSSVFFLTWMSFDRYIALARAMRCSLFRTKHHARLSCGLIWMASVSATLVPFTAVHLQHTDEA
CFCFADVREVQWLEVTLGFIVPFAIIGLCYSLIVRVLVRAHRHRGLRPRRQKALRMILAVVLVFFVCW
LPENVFISVHLLQRTQPGAAPCKQSFRHAHPLTGHIVNLAAFSNSCLNPLIYSFLGETFRDKLRLYIE
QKTNLPALNRFCHAALKAVIPDSTEQSDVRFSSAV.

Statistics. Linear and nonlinear regression analyses for all receptor binding assays and calculations of Kd and binding capacity were performed using GraphPad Prism for Windows (version 3.02; Graph Pad Software, San Diego, Calif.). Student's paired t test was used for paired comparisons and one-way ANOVA and Tukey tests for multiple comparisons (sigma Stat, SPss, Chicago, Ill.).

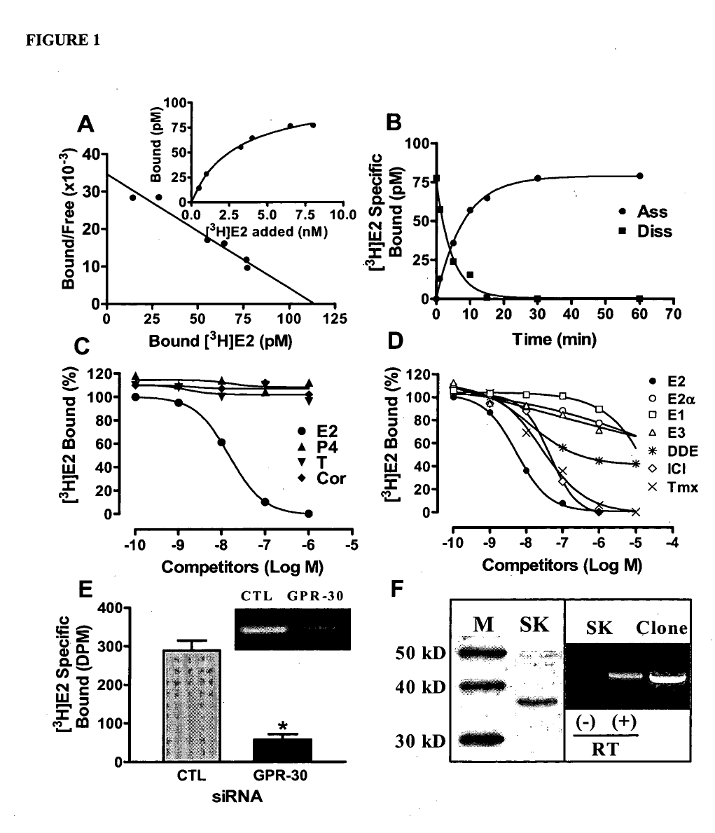

FIGS. 1A to 1F show the estrogen binding characteristics of plasma membranes from SKBR2 cells (ERα−, ERβ−, GPR30+). FIG. 1A is a representative saturation curve and Scatchard plot of specific [³H]-E2 binding. FIG. 1B is a time course of association (Ass) and dissociation (Diss) of specific [3H]-E2 binding. FIG. 1C shows competitive curves of steroid binding expressed as a percentage of maximum specific [3H]-E2 binding; E2, estradiol-17β; T, testosterone; P4, progesterone; Cor, cortisol. FIG. 1D shows competition curves of binding by estrogens. E2α, estradiol-17α; E1, estrone; E3, estriol; ICI, ICI 182, 780; Tmxf, tamoxifen; DDE, o,p′-DDE. FIG. 1E shows the effects of transfection with 100 nm GPR30 siRNA (GPR siRNA) on specific [³H]-E2 binding to cell membranes 18 hrs later; CTL: nonspecific control siRNA. Insert RT-PCR results. FIG. 1F shows the detection of GPR30 protein in SKBR3 (SK) cell membranes by Western blot analysis and GPR30 mRNA in cells by RT-PCR. M: protein molecular weight standards; clone: control GPR30 plasmid; (−) RT: lacking reverse transcriptase. (N=6,*P<0.05, Student's t test).

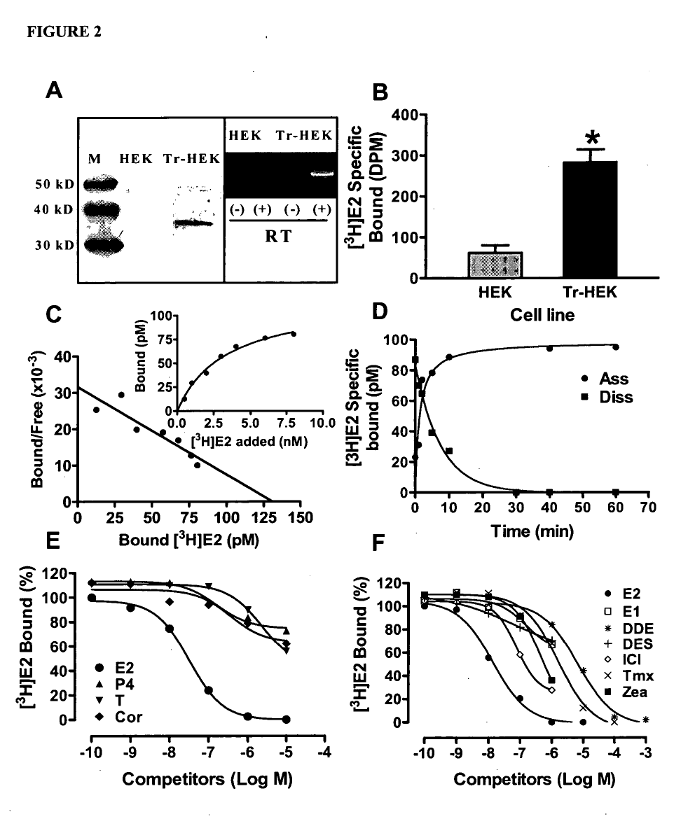

FIGS. 2A to 2E show that estrogen binds to plasma membranes of HEK293 cells (ERα−, ERβ−) stably transfected with GPR30. FIG. 2A shows the detection of GPR30 protein in transfected (Tr-HEK) cell membranes by Western blot analysis and GPR30 mRNA in cells by RT-PCR. HEK: untransfected control HEK293 cells. FIG. 2B is a single point assay of specific [³H]-E2 binding to cell membranes of HEK293 cells and of cells transfected with GPR30 (see key in FIG. 2A). FIG. 2C shows representative saturation curve and Scatchard plot of specific [³H]-E2 binding to membranes of transfected cells. FIG. 2D is a time course of association and dissociation of specific [³H]-E2 binding. FIG. 2E shows the competition curves of steroid binding. FIG. 2F shows competition curves of estrogen binding. DES, diethylstilbestrol; Zea, zearalanone (see FIG. 1 for key for other steroid abbreviations). (N=6,*P<0.05, Student's t test).

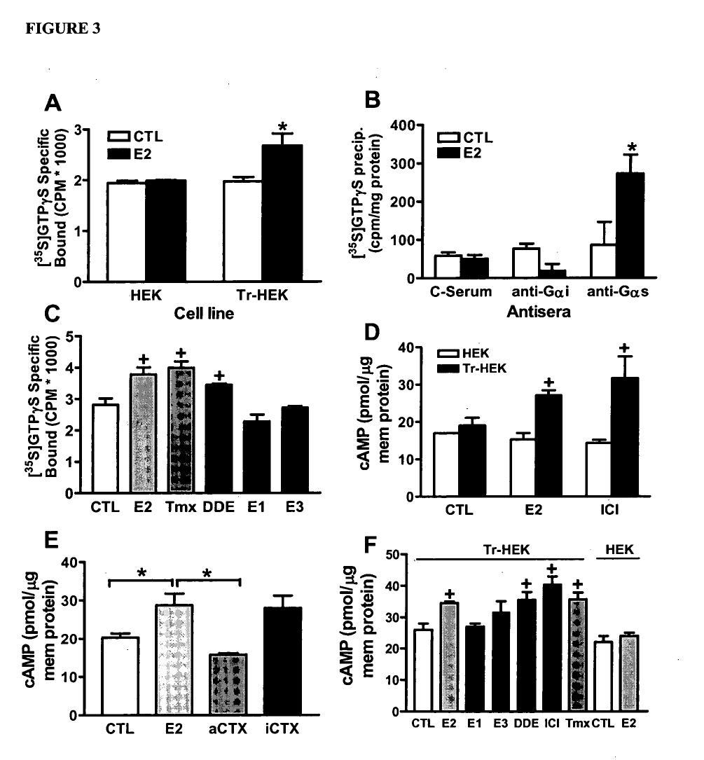

FIGS. 3A to 3F show the coupling of GPR30 to G-proteins and activation of adenylyl cyclase in SKBR3 and transfected HEK293 cells. FIG. 3A shows the effects of 20 min treatment with E2 (100 nm) on specific [³⁵S]GTPγS binding to G-proteins in membranes of transfected (Tr-HEK) and untransfected (HEK) HEK293 cells. FIG. 3B is an Immunoprecipitation of [³⁵S]GTPγS bound to G-proteins transfected with specific Gas (anti-Gas) and Gαᵢ (anti- Gαᵢ) G-protein antibodies or control rabbit serum (C-serum). CTL, control untreated membranes. HEK293 cells were treated with E2 or media (CTL) prior to membrane solubilization. FIG. 3C shows the effects of 20 min treatment with various estrogenic compounds (100 nm) on specific [³⁵S]GTPγS binding to membranes of SKBR3 cells. FIG. 3D shows the effects of 20 min treatment with 100 nm E2 or ICI 182, 780 on cAMP production by transfected (Tr-HEK) and untransfected HEK293 cells. FIG. 3E shows the effects of 20 min pretreatment with 10 μg/ml cholera toxin (aCTX, active; iCTX inactivated) on cAMP production by transfected HEK293 cells in response to 100 nm E2. FIG. 3F shows the effects of 20 min treatment with various estrogenic compounds (100 nm) on cAMP production by transfected (TR-HEK) and untransfected HEK293 cells. (N=6.*P<0.05, Student's t test; †P<0.05, one-way ANOVA).

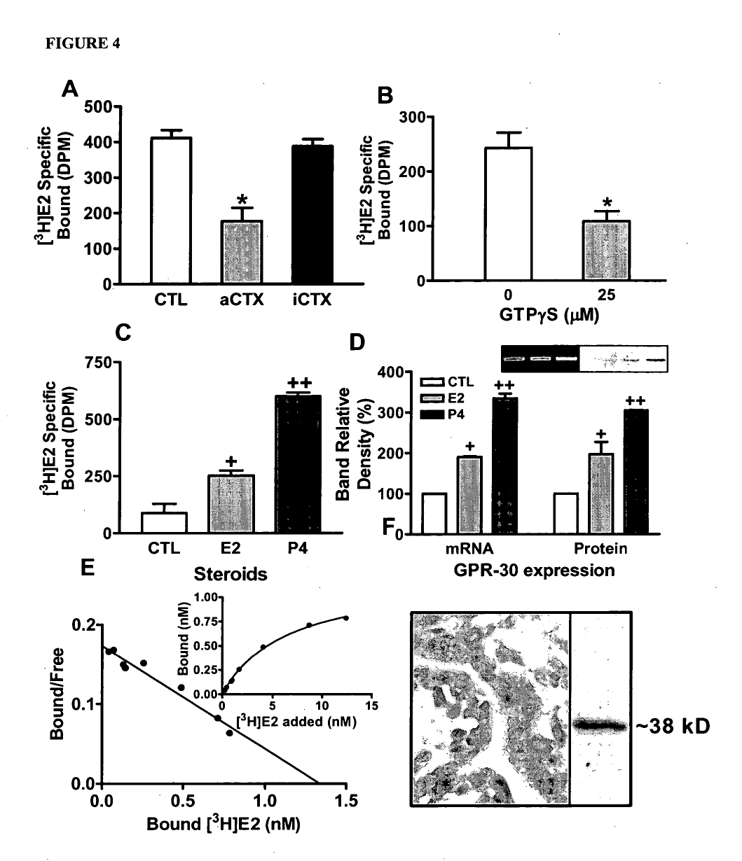

FIGS. 4A to 4F show the binding on E2. FIG. 4A shows the effects of pretreatment with 10 μg/ml cholera toxin (aCTX, active; iCTX inactivated) on specific[³H]-E2 binding to cell membranes of transfected HEK293 cells. FIG. 4B shows the effects of pretreatment with 25 μM GTPγS on specific[³H]-E2 binding to cell membranes of transfected HEK293 cells. FIG. 4C and FIG. 4D show the effects of 16 hr treatment of SKBR3 cells with 100 nm P4, E2 or media alone (CTL) on mER binding activity (FIG. 4C) and GPR30 mRNA and protein expression by semi-quantitative RT-PCR (FIG. 4D). FIG. 4E shows the Saturation curve and Scatchard plot of specific [³H]-E2 binding to human placenta cell membranes. FIG. 4F shows immunocytochemistry and Western blot analysis of human placental tissues and cell membranes, respectively, using a monoclonal GPR30 antibody. (N=6,*P<0.05, Student's t test; †P<0.05, ††P<0.001, one-way ANOVA).

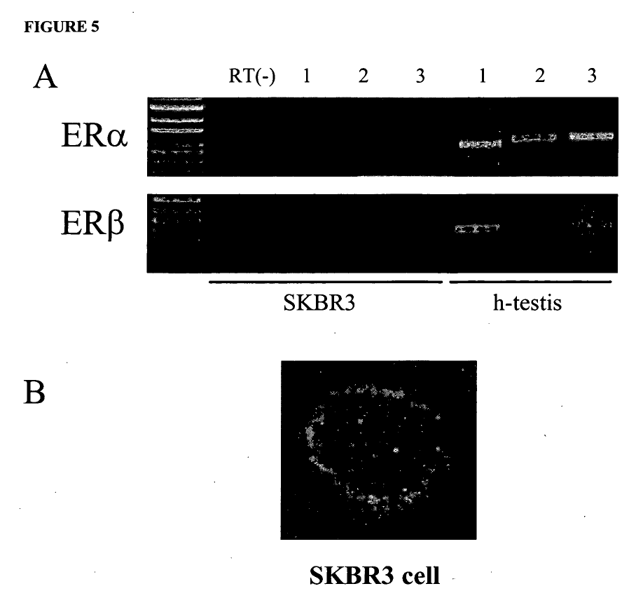

FIG. 5: Detection of nuclear estrogen receptors mRNA and protein in the SKBR3 cells. A. RT-PCR results, 1, 2, 3: human estrogen receptor α and β primer set 1, 2 and 3 (see details in supplementary text). B. Immunocytochemistry detection of GPR30 in the cell. The GPR30 protein was detected in the plasma membrane of the cell.

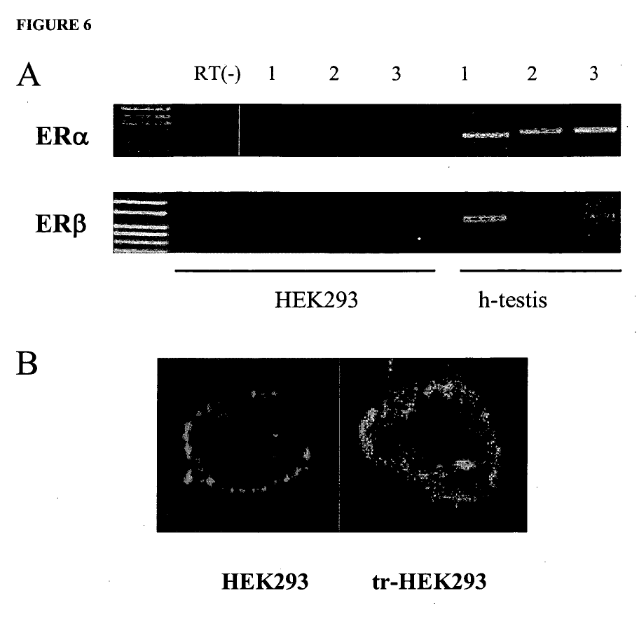

FIGS. 6A and 6B show the detection of nuclear estrogen receptorsmRNA and protein in the HEK293 cells. FIG. 6A shows RT-PCR results, 1, 2, 3: human estrogen receptor α and β primer set 1, 2 and 3 (see details in supplementary text). FIG. 6B shows the immunocytochemistry detection of GPR30 in transfected and non-transfected HEK293 cells. The GPR30 protein was detected in the plasma membrane of transfected cell, not in the non-transfected cell.

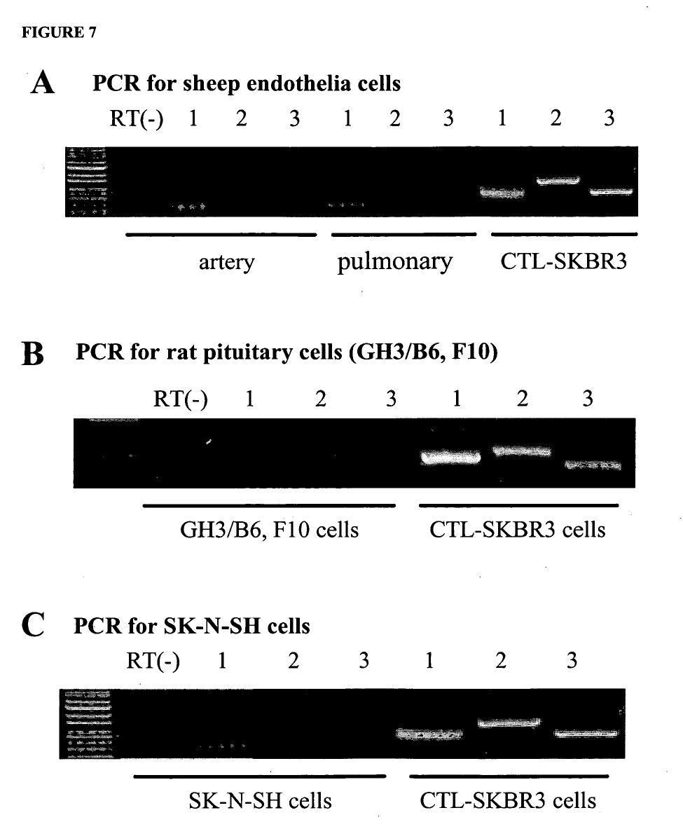

FIGS. 7A, 7B and 7C show the detection of GPR30 in estrogen responsive cells. FIG. 7A is an RT-PCR for sheep endothelia cells (artery and pulmonary cells), no expression was detected. FIG. 7B is an RT-PCR for rat pituitary cells (GK3/B6, F10), no expression was detected. FIG. 7C is an RT-PCR for SK-N-SH, no expression was detected. 1, 2, 3: GPR30 primer 1, 2 and 3.

Estrogen binding to plasma membranes of SKBR3 breast cancer cells. Saturation analysis and Scatchard plotting of [³H]E2 binding to SKBR3 cell plasma membranes showed the presence of a single, high affinity (Kd: 2.7 nM), saturable, low capacity (Bmax: 114 pM) specific estrogen binding site (FIG. 1 A). The binding was displaceable and the kinetics of association and dissociation of binding were rapid, with t_{1/2}s of 5.5 min and 8.1 min, respectively (FIG. 1 B). Competitive binding assays showed that steroid binding was specific for E2; P4, cortisol, and testosterone had no affinity for the receptor at concentrations up to 1 μM (FIG. 1 C). As observed previously for nERs and other estrogen membrane receptors, the inactive estradiol isomer, 17α-estradiol, failed to significantly displace E2 binding (FIG. 1 D). An unexpected finding was that the other natural estrogens, estriol and estrone had very low affinities for the receptor, less than 0.1% that of E2. In contrast, ICI 182, 780 and tamoxifen were effective competitors, with relative binding affinities (RBAs), approximately 10% that of E2. Interestingly, comparatively low concentrations (0.1-1 μM) of the xenoestrogen o,p′-DDE also caused significant displacement of [³H]-E2 binding. Treatment of the cells with GPR30 siRNA caused decreased expression of GPR30 mRNA that was accompanied by an 80% decrease in specific [³H]E2 membrane binding (FIG. 1 E). A major immunoreactive band was detected in plasma membranes of SKBR3 cells by Western blotting and GPR30 mRNA was detected in SKBR3 cells by RT-PCR (FIG. 1 F). Immunocytochemical analysis of whole SKBR3 cells demonstrates that GPR30 protein is concentrated at the cell periphery, consistent with its function as a membrane receptor (see FIG. 5B). The absence of ERα and ERβ mRNA in SKBR3 cells was confirmed by PCR using 3 sets of specific primers for each membrane (FIG. 5A).

Estrogen binding to plasma membranes of HEK293 cells transfected with GPR30. HEK293 cells do not express GPR30 mRNA and protein (FIG. 2A) or ERα, ERβ mRNA (FIG. 6) as shown by RT-PCR and Western blot analysis. Therefore, the ability to specifically bind E2 upon transfection with a cDNA encoding GPR30 was investigated. Expression levels of GPR30 mRNA and protein in the transfected HEK293 cells and membranes were similar to those in SKBR3 cells (FIG. 2A). Significant amounts of specific estrogen binding were detected in plasma membranes of cells transfected with GPR30, whereas negligible specific binding was detected in the plasma membranes of untransfected cells (FIG. 2B). Saturation analysis showed high affinity (Kd 3.3 nM), saturable (Bₘₐₓ 100 pM) specific [³H]E2 binding, and the Scatchard plot indicated a single binding site (FIG. 2C). The kinetics of association/dissociation of [³H]E2 binding to the recombinant protein produced in HEK393 cells were rapid with t_{1/2}s of 1.3 and 4.9 min (FIG. 2D). Competitive binding studies showed that binding was specific for E2 and certain estrogens. Estrone, diethystilbestrol and nonestrogenic compounds failed to significantly displace [³H]E2 at concentrations up to 10 μM, whereas tamoxifen, ICI 182, 780, o,p′-DDE and the mycotoxin estrogenic compound, zearalonone, displayed significant binding affinity (FIG. 2E,F). Immunocytochemical analysis showed that GPR30 was only detected on the plasma membranes of transfected whole cells (FIG. 6).

Activation of signal transduction pathways. Co-incubation with 100 nm E2 caused a significant increase in specific [³⁵S]GTPγS binding to membranes from HEK293 cells transfected with GPR30, but not to membranes of untransfected cells (FIG. 3A), whereas estradiol-17α treatment was ineffective (data not included). Immunoprecipitation of the membrane-bound [³⁵S]GTPγS with specific G-protein α subunit antibodies showed that the majority of the GTPγS is bound to the αₛ, subunit upon E2 treatment (FIG. 3B), which suggests the receptor activates a stimulatory G-protein (Gₛ). Treatment with 100 nm E2 caused a similar increase in specific [³⁵S]GTPγS binding to membranes of SKBR3 cells expressing wild-type GPR30, whereas estrone and estriol, which display low binding affinities for the mER in this cell line, failed to activate the G-proteins (FIG. 3C). Two other compounds with higher RBAs for the mER, tamoxifen and o,p′-DDE, also significantly increased [³⁵S]GTPγS binding to SKBR3 cell membranes, suggesting they mimic the actions of E2 on this nonclassical signaling pathway. Adenylyl cyclase activity, measured as an increase in cAMP content, was significantly increased in transfected HEK293 cells after 15 min treatment with 200 nm E2 and ICI 182, 780 but not in untransfected cells (FIG. 3D), in agreement with previous findings in SKBR3 cells (26). Moreover, the estrogen-induced increase in cAMP concentrations was blocked by prior treatment with activated cholera toxin (FIG. 3E), which is consistent with coupling of GPR30 to a stimulatory G-protein and activation of this pathway. Estrone and estriol did not alter cAMP production, but other compounds with higher RBAs for the mER in transfected cells, tamoxifen, ICI 182, 780 and o,p′-DDE significantly increased cAMP (FIG. 3F).

Modulation of estrogen binding to membranes. There was more than a 50% decrease in [³H]E2 binding to membranes of transfected cells after pretreatment with either cholera toxin or GTPγS (FIGS. 4A, 4B), that cause uncoupling of G-proteins from their receptors. Plasma membranes of SKBR3 cells cultured for 2 days in FBS-free media had low amounts of specific E2 binding. Pretreatment of the cells with 200 nm progesterone or E2 for 16 hrs in FBS-free media caused dramatic 9-fold and 2.5-fold increases in receptor binding, respectively, which paralleled the increases in GPR30 mRNA and protein expression (FIGS. 4C, 4D). Saturation analysis and Scatchard plotting also showed the presence of a high affinity (Kd 6.3 nM) limited capacity (Bmax 1.4 nM) single E2 binding site on human placental plasma membranes (FIG. 4E). Immunocytochemistry and Western blots showed specific GPR30 staining of human placenta epithelial cells and a 38 kD protein band, respectively (FIG. 4F).

Absence of GPR30 in several estrogen-responsive cells. GPR30 mRNA was not detected by RT-PCR in three well characterized models of non-classical E2 action, sheep endothelial cells (9), and in rat pituitary (GH₃/B6, F 10, 8) and hypothalamic (SK-N-SH, 31) cells (FIG. 7).

Discussion. The steroid binding and signal transduction experiments clearly demonstrate that GPR30 has all the characteristics of mERs that distinguish them from other previously described estrogen binding moeities. The finding that the plasma membranes of SKBR3 cells lacking ERα and ERβ (32; FIG. 5) but expressing GPR30 (24), show high affinity, limited capacity, displaceable, specific binding to E2 suggests the presence of a previously unknown estrogen receptor in these cells. The orphan GPCR-like protein, GPR30, is a candidate for a novel estrogen receptor because it is expressed and is an requisite signaling intermediary in estrogen-dependent activation of adenylyl cyclase and EGFR in SKBR3 in breast cancer cells that lack known ERs (FIG. 5; 24-26). Evidence in support of this hypothesis was obtained from experiments showing that both P4-induced increases and siRNA-induced decreases in GPR30 expression in SKBR3 cells were accompanied by parallel changes in specific E2 binding. Moreover, the observation that a tissue that expresses GPR30, human placenta (19), also shows similar high affinity E2 binding in its plasma membranes, suggests GPR30 is a functional mER in human tissues. Direct evidence that GPR30 binds estrogens and has the signal transduction characteristics of a mER was obtained from the studies with transfected HEK293 cells, which lack both nuclear ERα and ERβ (FIG. 6; 33). Untransfected HEK293 cells showed negligible E2 binding and E2 activation of G-proteins in their membrane fractions. However, plasma membranes of cells transfected with GPR30 displayed specific estrogen binding almost identical to that of the SKBR3 cells and characteristic of mERs identified previously (20-25). The steroid binding characteristics of the recombinant GPR30, like those of the wild type receptor, fulfill all the criteria for its designation as an mER. Both forms of GPR30 display high affinity and saturable E2 binding with Kds of ˜3.0 nM, similar to the affinities of other mERs (28). E2 consistently occupies a single binding site in cell and tissue membrane preparations as shown in the Scatchard plots. Moreover, E2 readily dissociates from the binding site, a critical feature of steroid receptors. The kinetics of association and dissociation were rapid, with t_{1/2}s<10 min, which is characteristic of membrane steroid receptors (27, 29). In addition, it was demonstrated that GPR30 acts as a mER in transfected cells to transduce the signals of estrogenic compounds with high RBAs for the receptor, resulting in activation of a stimulatory G-protein and upregulation of adenylyl cyclase activity, whereas estriol, estrone, which had low RBAs for the receptor, were inactive. Finally, the decrease in mER binding in transfected cell membranes observed after treatment with agents causing uncoupling of G-proteins from GPCRs, GTPγS and CTX (30, 34), indicates the mER is directly coupled to G-protein and is a GPCR, consistent with its identity as GPR30. This is the first report of a protein structurally unrelated to nuclear estrogen receptors that has the characteristics of an estrogen receptor. The discovery of this novel mER provides a plausible mechanism by which estrogens can initiate rapid steroid actions at the cell surface and act in certain nER negative target cells. The existence of this mER-mediated signaling pathway also explains some of the pleiotropic actions of estrogens in breast and other estrogen target tissues.

The identification of a novel mER that activates a stimulatory G-protein (Gs) indicates that estrogen and anti-estrogen signaling in human breast cancer is more complex than previously recognized. Several characteristics of the receptor have important implications for the development of the disease. Estrogens can activate pathways involved in proliferative responses, such as MAPkinase via epidermal growth factor receptor (EGFR) transactivation, and c- fos expression, in nER-negative breast cancer cells via GPR30 (24, 35). Recent studies show that Gₛ-coupled GPCRs, in addition to G_{q}-coupled ones, can stimulate EGFR transactivation (36). GPR30 transactivates the EGFR by release of HB-EGF from the cell surface by a Gβγ-Src-Shc signaling pathway (26). Gₛ-coupled receptors can signal to Src and Shc via β-arrestin scaffolds (37), and this could provide an alternative mechanism by which they transactivate EGFR. The finding that estrogen also attenuates the EGFR-to-ERK signaling axis by cAMP-dependent signaling (25) via GPR30 indicates an additional role of this novel receptor in regulating EGF action. Interestingly, GPR30 is expressed abundantly in human primary breast carcinomas and breast cancer cells lines that are nER positive, but shows no or minimal expression in ER-negative breast cancer tissues and cells (19). The finding that GPR30 is upregulated by P4 confirms the results of a previous study (38) and indicates a probable mechanism of co-ordinate hormonal control of GPR30 and the nERs. Environmental contaminants that are weak nER agonists (xenoestrogens), such as PCBs and the DDT derivative, o,p′-DDE, have also been implicated in tumorigenesis in breast and other estrogen target tissues, presumably via nER activation (39-40). The finding that o,p′-DDE is an agonist for GPR30 receptor activity demonstrates that xenoestrogens can also activate this alternative estrogen signaling pathway in breast cancer cells, as has been shown for mERs in other tissues (14,27). Interestingly, the RBA of o,p′-DDE binding to GPR30 and nERs are similar (41), possibly indicating a similar susceptibility of these two estrogen signaling pathways to interference by this xeonestrogen. Interference with nontraditional steroid actions by xenoestrogen binding to steroid membrane receptors has previously demonstrated for the mPR on fish gametes (42). The present results extend this novel mechanism of endocrine disruption to a second GPCR-like steroid receptor and suggest that interactions of xenoestrogens with ligand binding sites is a shared feature of both nuclear and GPCR-like steroid receptors.

The results also have profound implications for the treatment of breast cancer. Patients treated for ER-positive breast cancers are frequently administered the antiestrogen tamoxifen to prevent reoccurrence of tumor growth (43). However, our results show that tamoxifen and the nER antagonist ICI 182, 780 have opposite actions on the alternative mER-mediated pathway, acting as estrogen agonists by binding to GPR30 and activating G-proteins. Once the GCPR estrogen binding was demonstrated, the agonist activity of the pure ER antagonist ICI 182, 780 was expected, since it was found that GCPR has a high RBA for GPR30 and it has previously been shown to mimic estrogen actions initiated at the cell membrane in a broad range of targets, including SKBR3 cells (25, 28). The identification of GPR30 as a mER facilitates investigations on its role in the physiology and pathology of breast, prostate, placenta, ovarian, neural and vascular tissues and also provides current target and methods for screening and isolating one or more agents for therapeutic intervention.

The discovery of a second class of estrogen receptors unrelated to nERs provides an entirely new model to explore the structural requirements for estrogen binding and activation of receptor proteins. The marked differences in the RBAs of some estrogens to GPR30 and their affinities to ERα and ERβ and a third distinct ER subtype in fishes was expected, considering the lack of structural similarity between GPR30 and the nERs. Initial binding studies with a limited number of nER ligands suggests GPR30 has a higher specificity for estradiol-17β binding than the nERs; all the other estrogens tested had RBAs of 10% or lower for the membrane receptor. Interestingly, the presence of other functional groups on the D ring of the steroid molecule in the vicinity of the 17 position or alteration of the 17β OH configuration dramatically decreases binding to GPR30, estrone, estriol and estradiol-17α having RBAs less than 1% that of estradiol-17β, whereas these changes result in relatively modest decreases in binding affinity to the nERs. In contrast, alteration of the four carbon ring structure to produce tamoxifen or addition of a large side chain at the 7 position to produce ICI 182, 780 caused only minor decreases in RBA to GPR30, similar to that observed with some nERs.

Although the identities of mERs remains uncertain and topic of intense debate, there is a growing body of evidence indicating a role for nuclear ER or ER-like proteins in many tissues showing rapid, cell surface-mediated estrogen actions (7-12). The absence of GPR30 in several well-characterized cell models of rapid, nongenomic estrogen actions, sheep endothelial cells, rat pituitary and hypothalamic cells (FIG. 7; 8,9, 31), suggests that not all these estrogen actions are mediated via GPR30 and that at least two classes of mERs are present in vertebrates. The physiological significance of the presence of both types of mERs in certain cell types, such as MCF-7 cells is unclear (35, 44). However, the Kd of E2 binding to membranes of SKBR3 and HEK293 cells expressing GPR30 in the present study ranged from 2.7-3.3 nm, 10 fold higher than that reported for membranes of CHO cells transfected with ERα (7), and may be indicative of a higher threshold concentration for activation of GPR30-dependent signaling pathways by estrogens.

Despite the sequence knowledge of GPR30 and mPRs, the current invention for the first time isolated, identifies and characterizes two distinct classes of GPCRs with no sequence homology and few apparent structural similarities. Thus, there is no indication that these mERs and mPRs arose from a common ancestor, unlike members of the nuclear steroid receptor superfamily (45). The C-terminal domain of GPR30 is longer than that of the mPRs (47 vs 12 amino acids), the DRY sequence involved in signal transduction in intracellular loop 2 is absent in the mPRs, whereas the length of the second extracellular loop in GPR30 (10-20 amino acids) is shorter than that of the mPRs (˜50 amino acids). On the other hand both receptors have seven transmembrane domains, N-terminal glycosylation sites and two conserved cysteines in the first two extracellular loops which can form disulphide bonds to help stabilize the structure, basic features of GPCRs (19, 20). In addition, both receptors have large N-terminal extracellular domains, 57-75 amino acids long, that could possibly be involved in ligand binding. The discovery of two apparently unrelated families of GPCR-like membrane steroid receptors raises interesting evolutionary questions regarding their origins, such as whether the ancestral proteins were receptors for nonsteroidal ligands that subsequently acquired new functions (neofunctionalization) to bind and transduce specific steroid signals, and if so whether the receptors have retained their responses these nonsteroidal ligands. Information on the tissue distribution, regulation and ligand specificity of these receptors should provide insights into the evolution and functions of this new class of steroid receptors.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Revelli A, Massobrio M, Tesarik J 1998 Nongenomic actions of steroid hormones in reproductive tissues. Endocr Rev 19:3-17
2. Falkenstein E, Tillmann H C, Christ M, Feuring M, Wehling M 2000 Multiple actions of steroid hormones—a focus on rapid, nongenomic effects. Pharmacol Rev 52:513-56
3. Norman AW, Mizwicki M T, Norman D P 2004 Steroid-hormone rapid actions, membrane receptors and a conformational ensemble model. Nat Rev Drug Discov 3:27-41
4. Nadal A, Ropero A B, Fuentes E, Soria B 2001 The plasma membrane estrogen receptor: nuclear or unclear? Trends Pharmacol Sci 22:597-9
5. Watson C S, Zivadinovic D, Bulayeva N, Hawkins B, Campbell C, Gametchu B 2003 A membrane form of estrogen receptor-α mediates estrogenic, nongenomic effects. In: Watson C S eds. The identities of membrane steroid receptors and other proteins mediating nongenomic steroid action. Boston/Dordrecht/London: Kluwer Academic Publishers; 11-9
6. Bramley T 2003 Non-genomic progesterone receptors in the mammalian ovary: some unresolved issues. Reproduction 125:3-15
7. Razandi M, Pedram A, Greene G L, Levin E R 1999 Cell membrane and nuclear estrogen receptors (ERs) originate from a single transcript: studies of ERalpha and ERbeta expressed in Chinese hamster ovary cells. Mol Endocrinol 13:307-19
8. Watson C S, Norfleet A M, Pappas T C, Gametchu B 1999 Rapid actions of estrogens in GH3/B6 pituitary tumor cells via a plasma membrane version of estrogen receptor-alpha. Steroids 64:5-13
9. Chen Z, Yuhanna I S, Galcheva-Gargova Z, Karas R H, Mendelsohn M E, Shaul P W 1999 Estrogen receptor alpha mediates the nongenomic activation of endothelial nitric oxide synthase by estrogen. J Clin Invest 103:401-6
10. Song R X, McPherson R A, Adam L, et al. 2002 Linkage of rapid estrogen action to MAPK activation by ERalpha-Shc association and Shc pathway activation. Mol Endocrinol 16:116-27
11. Monje P, Boland R 1999 Characterization of membrane estrogen binding proteins from rabbit uterus. Mol Cell Endocrinol 147:75-84
12. Qiu J, Bosch M A, Tobias S C, et al. 2003 Rapid signaling of estrogen in hypothalamic neurons involves a novel G-protein-coupled estrogen receptor that activates protein kinase C. J Neurosci 23:9529-40
13. Benten W P, Stephan C, Lieberherr M, Wunderlich F 2001 Estradiol signaling via sequestrable surface receptors. Endocrinology 142:1669-77
14. Nadal A, Ropero A B, Laribi O, Maillet M, Fuentes E, Soria B 2000 Nongenomic actions of estrogens and xenoestrogens by binding at a plasma membrane receptor unrelated to estrogen receptor alpha and estrogen receptor beta. Proc Natl Acad Sci USA 97:11603-8
15. Sylvia V L, Boyan B D, Dean D D, Schwartz Z 2000 The membrane effects of 17beta-estradiol on chondrocyte phenotypic expression are mediated by activation of protein kinase C through phospholipase C and G-proteins. J Steroid Biochem Mol Biol 73:211-24
16. Gu Q, Korach K S, Moss R L 1999 Rapid action of 17beta-estradiol on kainate-induced currents in hippocampal neurons lacking intracellular estrogen receptors. Endocrinology 140:660-6
17. Zhu Y, Rice C D, Pang Y, Pace M, Thomas P 2003 Cloning, expression, and characterization of a membrane progestin receptor and evidence it is an intermediary in meiotic maturation of fish oocytes. Proc Natl Acad Sci USA 100:2231-6
18. Zhu Y, Bond J, Thomas P 2003 Identification, classification, and partial characterization of genes in humans and other vertebrates homologous to a fish membrane progestin receptor. Proc Natl Acad Sci USA 100:2237-42
19. Carmeci C, Thompson D A, Ring H Z, Francke U, Weigel R J 1997 Identification of a gene (GPR30) with homology to the G-protein-coupled receptor superfamily associated with estrogen receptor expression in breast cancer. Genomics 45:607-17
20. Owman C, Blay P, Nilsson C, Lolait S J 1996 Cloning of human cDNA encoding a novel heptahelix receptor expressed in Burkitt's lymphoma and widely distributed in brain and peripheral tissues. Biochem Biophys Res Commun 228:285-92
21. O'Dowd B F, Nguyen T, Marchese A, et al. 1998 Discovery of three novel G-protein-coupled receptor genes. Genomics 47:310-3
22. Takada Y, Kato C, Kondo S, Korenaga R, Ando J 1997 Cloning of cDNAs encoding G protein-coupled receptor expressed in human endothelial cells exposed to fluid shear stress. Biochem Biophys Res Commun 240:737-41
23. Feng Y, Gregor P 1997 Cloning of a novel member of the G protein-coupled receptor family related to peptide receptors. Biochem Biophys Res Commun 231:651-4
24. Filardo E J, Quinn J A, Bland K I, Frackelton A R, Jr. 2000 Estrogen-induced activation of Erk-1 and Erk-2 requires the G protein-coupled receptor homolog, GPR30, and occurs via trans-activation of the epidermal growth factor receptor through release of HB-EGF. Mol Endocrinol 14:1649-60
25. Filardo E J, Quinn J A, Frackelton A R, Jr., Bland K I 2002 Estrogen action via the G protein-coupled receptor, GPR30: stimulation of adenylyl cyclase and cAMP-mediated attenuation of the epidermal growth factor receptor-to-MAPK signaling axis. Mol Endocrinol 16:70-84
26. Filardo E J 2002 Epidermal growth factor receptor (EGFR) transactivation by estrogen via the G-protein-coupled receptor, GPR30: a novel signaling pathway with potential significance for breast cancer. J Steroid Biochem Mol Biol 80:231-8
27. Loomis A K, Thomas P 2000 Effects of estrogens and xenoestrogens on androgen production by Atlantic croaker testes in vitro: evidence for a nongenomic action mediated by an estrogen membrane receptor. Biol Reprod 62:995-1004
28. Patino R, Thomas P 1990 Characterization of membrane receptor activity for 17 alpha, 20 beta, 21-trihydroxy-4-pregnen-3-one in ovaries of spotted seatrout (Cynoscion nebulosus). Gen Comp Endocrinol 78:204-17
29. Braun A M, Thomas P 2004 Biochemical characterization of a membrane androgen receptor in the ovary of the atlantic croaker (Micropogonias undulatus). Biol Reprod 71:146-55
30. Liu D, Dillon J S 2002 Dehydroepiandrosterone activates endothelial cell nitric-oxide synthase by a specific plasma membrane receptor coupled to Galpha (i2,3). J Biol Chem 277:21379-88
31. Watters J J, Campbell J S, Cunningham M J, Krebs E G, Dorsa D M 1997 Rapid membrane effects of steroids in neuroblastoma cells: effects of estrogen on mitogen activated protein kinase signalling cascade and c-fos immediate early gene transcription. Endocrinology 138:4030-3
32. Vladusic E A, Hornby A E, Guerra-Vladusic F K, Lakins J, Lupu R 2000 Expression and regulation of estrogen receptor beta in human breast tumors and cell lines. Oncol Rep 7:157-67
33. Kahlert S, Nuedling S, van Eickels M, Vetter H, Meyer R, Grohe C 2000 Estrogen receptor alpha rapidly activates the IGF-1 receptor pathway. J Biol Chem 275:18447-53
34. Cunha R A, Constantino M D, Ribeiro J A 1999 G protein coupling of CGS 21680 binding sites in the rat hippocampus and cortex is different from that of adenosine A1 and striatal A2A receptors. Naunyn Schmiedebergs Arch Pharmacol 359:295-302
35. Maggiolini M, Vivacqua A, Fasanella G, et al. 2004 The G protein-coupled receptor GPR30 mediates c-fos up-regulation by 17beta-estradiol and phytoestrogens in breast cancer cells. J Biol Chem 279:27008-16
36. Bertelsen L S, Barrett K E, Keely S J 2004 Gs protein-coupled receptor agonists induce transactivation of the epidermal growth factor receptor in T84 cells: implications for epithelial secretory responses. J Biol Chem 279:6271-9
37. Luttrell L M, Ferguson S S, Daaka Y, et al. 1999 Beta-arrestin-dependent formation of beta2 adrenergic receptor-Src protein kinase complexes. Science 283:655-61
38. Ahola T M, Purmonen S, Pennanen P, Zhuang Y H, Tuohimaa P, Ylikomi T 2002 Progestin upregulates G-protein-coupled receptor 30 in breast cancer cells. Eur J Biochem 269:2485-90
39. Tsuda H, Naito A, Kim C K, Fukamachi K, Nomoto H, Moore M A 2003 Carcinogenesis and its modification by environmental endocrine disruptors: in vivo experimental and epidemiological findings. Jpn J Clin Oncol 33:259-70
40. Birnbaum L S, Fenton S E 2003 Cancer and developmental exposure to endocrine disruptors. Environ Health Perspect 111:389-94
41. Loomis A K, Thomas P 1999 Binding characteristics of estrogen receptor (ER) in Atlantic croaker (Micropogonias undulatus) testis: different affinity for estrogens and xenobiotics from that of hepatic ER. Biol Reprod 61:51-60
42. Das S, Thomas P 1999 Pesticides interfere with the nongenomic action of a progestogen on meiotic maturation by binding to its plasma membrane receptor on fish oocytes. Endocrinology 140:1953-6
43. Gelber R D, Bonetti M, Castiglione-Gertsch M, Coates A S, Goldhirsch A 2003 Tailoring adjuvant treatments for the individual breast cancer patient. Breast 12:558-68
44. Razandi M, Pedram A, Park S T, Levin E R 2003 Proximal events in signaling by plasma membrane estrogen receptors. J Biol Chem 278:2701-12
45. Thornton J W 2001 Evolution of vertebrate steroid receptors from an ancestral estrogen receptor by ligand exploitation and serial genome expansions. Proc Natl Acad Sci USA 98:5671-6.

1. A composition comprising an isolated and purified G-protein-coupled receptor that binds specifically to an estrogen.

2. The composition of claim 1, wherein the G-protein receptor comprises GPR30.

3. The composition of claim 1, wherein the G-protein receptor comprises human GPR30.

4. The composition of claim 1, wherein the isolated G-protein receptor polypeptide is a fusion protein.

5. The composition of claim 1, wherein the isolated G-protein receptor polypeptide is a fusion protein comprising a myc-tag, a His-tag, FLAG-tag, Glutathione-S-Transferase, Maltose-Binding Protein or combinations thereof.

6. The composition of claim 1, wherein the G-protein receptor triggers non-classical estrogen signaling.

7. The composition of claim 1, wherein the G-protein receptor binds to estradiol-17β (E2).

8. The composition of claim 1, wherein the G-protein receptor is isolated from neural, breast cancer, placental, heart, ovarian, prostate, hepatic, vascular epithelial and lymphoid tissues.

9. An isolated polypeptide encoded by the nucleic acid fragment of SEQ ID NO.:1, GenBank; accession No. BC011634.

10. A method of identifying a test compound that modulates the binding of an estrogen to a G-protein-coupled receptor comprising the step of measuring binding of an estrogen to a G-protein receptor to the estrogen in the presence and absence of a test compound, wherein the test compound modulates the binding of the estrogen to the G-protein receptor and is indicative that the test compound is a modulator of the binding of the estrogen to the G-protein receptor.

11. The method of claim 10, wherein the steroid is selected from 17β-estradiol (E2), 17α-estradiol (E2α), estrone (E2), estriol (E3), cortisol (cor), testosterone (T) and progesterone (P4) and the synthetic estrogen diethylstilbestrol (DES).

12. The method of claim 10, wherein the test compound is tamoxifen (Tmx), zearalenone, dichlorodiphenyltrichloroethane, o,p′-DDE (DDE), the synthetic antiestrogen ICI 182, 780, a taxol-derivative or salts thereof.

13. A vector comprising the nucleic acid sequence of SEQ ID NO.:1, GenBank; accession No. BC011634.

14. A host cell comprising a vector comprising the nucleic acid sequence of SEQ ID NO.:1, GenBank; accession No. BC011634.

15. A method of treating cancer, comprising the steps of:
identifying a patient in need of cancer therapy; and
providing to the patient an effective dose of a GPCR modulator.

16. The method of claim 15, wherein the GPCR modulator comprises a GPCR agonist.

17. The method of claim 15, wherein the GPCR modulator comprises a GPCR antagonist.

18. The method of claim 15, wherein the GPCR modulator comprises inhibition of a GTPase activity of the GPCR.

19. The method of claim 15, wherein the GPCR modulator comprises activation of a GTPase activity of the GPCR.

20. A method of diagnostic a condition related to nonclassical estrogen binding, comprising the step of binding a GPCR binding agent comprising a detectable label to a cell.

21. The method of claim 20, wherein the GPCR binding agent is specific for membrane estrogen binding activity.

22. A dosage form comprising a therapeutically effective amount of an estrogen or estrogen derivative that is specific for a GPCR.

23. The dosage form of claim 22, further comprising an estrogen modulator.

24. The dosage form of claim 22, wherein the estrogen or estrogen derivative comprises a GPCR agonist.

25. The dosage form of claim 22, wherein the estrogen or estrogen derivative comprises a GPCR antagonist.

26. The dosage form of claim 22, wherein the estrogen or estrogen derivative inhibits a GTPase activity of the GPCR.

27. The dosage form of claim 22, wherein the estrogen or estrogen derivative activates a GTPase activity of the GPCR.

28. The dosage form of claim 23, wherein the estrogen modulator comprises an estrogen agonist.

29. The dosage form of claim 23, wherein the estrogen modulator comprises an estrogen antagonist.

30. A method of identifying a GCPR modulator comprising the steps of:
screening a compound library for one or more agents that bind to a membrane-associated G-protein estrogen receptor.

31. The method of claim 30, further comprising the step of determining if the one or more agents that bind to the membrane-associated G-protein receptor is selective for the membrane-associated G-protein estrogen receptor.

32. An isolated and purified membrane-associated G-protein estrogen receptor.

33. The receptor of claim 32, further defined as comprising a human GPR30.

34. A diagnostic method comprising the steps of:
characterizing the expression of an isolated and purified membrane-associated G-protein estrogen receptor of a patient, and
treating the patient with an agent that modifies the activity of the membrane-associated G-protein estrogen receptor.


SEQUENCE LISTING (8 sequences)

SEQ ID NO: 1 - DNA, 2218 bp, Homo sapiens
ggcacggaggctttctaaagatggattcaccatttaaaacagagctctgggagcctttcggcaaatcttgaaagctgcacggcgcagagacatggatgtgacttcccaagcccggggcgtgggcctggagatgtacctaggcaccgcgcagcctgcggcccccaacaccacctcccccgagctcaacctgtcccacccgctcctgggcaccgccctggccaatgggacaggtgagctctcggagcaccagcagtacgtgatcggcctgttcctctcgtgcctctacaccatcttcctcttccccatcggctttgtgggcaacatcctgatcctggtggtgaacatcagcttccgcgagaagatgaccatccccgacctgtacttcatcaacctggcggtggcggacctcatcctggtggccgactccctcattgaggtgttcaacctgcacgagcggtactacgacatcgccgtcctgtgcaccttcatgtcgctcttcctgcaggtcaacatgtacagcagcgtcttcttcctcacctggatgagcttcgaccgctacatcgccctggccagggccatgcgctgcagcctgttccgcaccaagcaccacgcccggctgagctgtggcctcatctggatggcatccgtgtcagccacgctggtgcccttcaccgccgtgcacctgcagcacaccgacgaggcctgcttctgtttcgcggatgtccgggaggtgcagtggctcgaggtcacgctgggcttcatcgtgcccttcgccatcatcggcctgtgctactccctcattgtccgggtgctggtcagggcgcaccggcaccgtgggctgcggccccggcggcagaaggcgctccgcatgatcctcgcggtggtgctggtcttcttcgtctgctggctgccggagaacgtcttcatcagcgtgcacctcctgcagcggacgcagcctggggccgctccctgcaagcagtctttccgccatgcccaccccctcacgggccacattgtcaacctcgccgccttctccaacagctgcctaaaccccctcatctacagctttctcggggagaccttcagggacaagctgaggctgtacattgagcagaaaacaaatttgccggccctgaaccgcttctgtcacgctgccctgaaggccgtcattccagacagcactgagcagtcggatgtgaggttcagcagtgccgtgtagacagccttggccacataggcccagccagggtgtgactcgggagctgcacacacctgggtggacacaaggcacggccacgtcatgtctctaaactgcggtcagatgtggcttctggctcctcggggcctcgcgagggtcacgcttgcctggtcaccctggggctgcttaggaaacctcacgactggtcaccttgcactcctcacacagaattgctacaatcccaaagcgctcgccccgcagggtccaaaggccagcggtgaccagcctgtcacccagctcctccccgccaaccctgcctgccgctgcacctgcctgccgctgcaggaaacatttctgacaccgtcgaccaggaaagccacacggagaggccactgtgggtgaagcgcctcagttacacaggaaccctaaagcaaatctgccaccgtgggggaactgacgctggagatgcaaggtgctggtgggtctgagctggacgtcgcggtgtgtcctctgtgcccacggtctgagctagctagcgcaccgccgagttaaagaggagaaggaaaacatgctgctctggtgcacgcctgagcgtcctccatcttccaggatggcagcaatggcgctgtgcggcctcaccaggcccacgaggagcagcagcgctcggcccggagcagcaggaaggcccctctgtggagcgcccgccgtctgctccggggtggttcagtcactgcttgttgacatcaacatggcaattgcactcatgtggactgggaccgtgcgagctgccgtgtgggttagtcgggtgccaggacaatgaaatactccagcacgtgtggctgacgaatttgtttctacagaaataacagctggggacaactgcggtgatgatgtaaaaaccttcccataaaatgtaagaaaagctgatgaggctggtgacgttcagcctttgtcaataaacctgtcatgtgcggataaaaaaaaaaaaaaaaaaaaaaaaaaaaaaaaaaaaaaaaaaaa

SEQ ID NO: 2 - protein, 375 aa, Homo sapiens
MDVTSQARGVGLEMYLGTAQPAAPNTTSPELNLSHPLLGTALANGTGELSEHQQYVIGLFLSCLYTIFLFPIGFVGNILILVVNISFREKMTIPDLYFINLAVADLILVADSLIEVFNLHERYYDIAVLCTFMSLFLQVNMYSSVFFLTWMSFDRYIALARAMRCSLFRTKHHARLSCGLIWMASVSATLVPFTAVHLQHTDEACFCFADVREVQWLEVTLGFIVPFAIIGLCYSLIVRVLVRAHRHRGLRPRRQKALRMILAVVLVFFVCWLPENVFISVHLLQRTQPGAAPCKQSFRHAHPLTGHIVNLAAFSNSCLNPLIYSFLGETFRDKLRLYIEQKTNLPALNRFCHAALKAVIPDSTEQSDVRFSSAV

SEQ ID NO: 3 - DNA, 18 bp, Artificial Sequence
  Primer
ggctttgtgggcaacatc

SEQ ID NO: 4 - DNA, 19 bp, Artificial Sequence
  Primer
cggaaagactgcttgcagg

SEQ ID NO: 5 - DNA, 20 bp, Artificial Sequence
  Primer
tggtggtgaacatcagcttc

SEQ ID NO: 6 - DNA, 20 bp, Artificial Sequence
  Primer
tgagcttgtccctgaaggtc

SEQ ID NO: 7 - DNA, 20 bp, Artificial Sequence
  Primer
gcagcgtcttcttcctcacc

SEQ ID NO: 8 - DNA, 20 bp, Artificial Sequence
  Primer
acagcctgagcttgtccctg